SEQUENCE LISTING (2 sequences)

SEQ ID NO: 1 - DNA, 36 bp, Artificial Sequence
  ChR2 forward primer
gcaggtcgactctagagccaccatggactatggcgg

SEQ ID NO: 2 - DNA, 43 bp, Artificial Sequence
  ChR2 reverse primer
tcattttttctaccggtaccgctggcacggctccggcctcggc

(19) United States
(12) Patent Application Publication
(10) Pub. No.: US 2017/0339930 A1
(43) Pub. Date: Nov. 30, 2017

(54) METHOD OF REGULATING LIFESPAN USING TRANSGENIC CAENORHABDITIS ELEGANS

(71) Applicant: POSTECH ACADEMY-INDUSTRY FOUNDATION, POHANG (KR)

(72) Inventors: SEUNG-JAE LEE, POHANG (KR); MURAT ARTAN, POHANG (KR); DAE-EUN JEONG, SEOUL (KR)

(21) Appl. No.: 15/596,049

(22) Filed: May 16, 2017

(30) Foreign Application Priority Data
May 24, 2016 (KR) 10-2016-0063225

(51) Int. Cl. A01K 67/033

(52) U.S. Cl. CPC A01K 67/0336; A01K 2267/0393; A01K 2267/03; A01K 2207/30; A01K 2217/072; A01K 2227/703

(57) ABSTRACT

The present disclosure relates to transgenic Caenorhabditis elegans including, in sensory neurons, Channelrhodopsin 2 (ChR2)::Green Fluorescence Protein (GFP) DNA in which the ChR2 gene and the GFP gene are linked, a method of producing the same, a method of regulating the lifespan thereof, and a method of screening an aging regulation candidate by using the same. The present disclosure may also provide an animal model for research into prevention/treatment of aging-related diseases by regulating the lifespan of an animal on a subject level and a method of screening a drug candidate for prevention/treatment of aging-related diseases.

### CROSS-REFERENCE TO RELATED APPLICATION

This application claims priority to and the benefit of Korean Patent Application No. 10-2016-0063225, filed on May 24, 2016, the disclosure of which is incorporated herein by reference in its entirety.

### BACKGROUND

### 1. Field of the Invention

The present disclosure relates to transgenic Caenorhabditis elegans including, in sensory neurons, Channelrhodopsin 2 (ChR2)::Green Fluorescence Protein (GFP) DNA in which the ChR2 gene and the GFP gene are linked, a method of producing the same, a method of regulating the lifespan thereof, and a method of screening an aging regulation candidate by using the same.

### 2. Discussion of Related Art

Caenorhabditis elegans is mainly found in soil and rotten fruits and lives by consuming various types of bacteria. Caenorhabditis elegans consists of about 1000 cells on the basis of the adult body, and certain types of cells form several tissues (e.g., nerves, intestines, muscles, skin tissue, and the like) each having a specific function. Caenorhabditis elegans has actively been used as model animals in aging genetics research due to its relatively simple biological system, high fertility, ease of gene manipulation, short lifespan (about 3 weeks), and the like. In addition, Caenorhabditis elegans has five pairs of homologous chromosomes and X chromosomes and it has been discovered that the number of genes encoding protein therein is about 20,000. About 40% of the genes are evolutionarily conserved in mammals including humans in terms of sequence and function. A considerable number of genes discovered to have an aging regulation function through Caenorhabditis elegans research have been reported to have similar functions on Drosophila, mice, and the like.

Caenorhabditis elegans has about 300 neurons and a connection map for all the neurons has been completed. As in other animals, neurons of Caenorhabditis elegans are classified into three types: sensory neurons, interneurons, and motor neurons. Sensory neurons perceive changes in external environmental signals and induce physiological and behavioral responses in the body of Caenorhabditis elegans according thereto.

In addition, it has been discovered that the sensory neurons of Caenorhabditis elegans also regulate lifespans on a subject level and, interestingly, the loss of function of such sensory neurons has been reported to lead to an increase in lifespan by approximately 30% to 40%. In this regard, it is known that the increase in lifespan due to sensory neuronal dysfunction requires the activity of DAF-16 (FOX( )transcription factor) which acts as a downstream signal of an insulin signaling pathway.

Genes of the insulin signaling pathway are essential signaling systems in development, immunity, aging regulation, and the like of subjects. When a mutation is induced in daf-2 (Insulin/Insulin-like Growth Factor type 1 (IGF-1) Receptor), it has been known that immunity and the lifespans of subjects increase. In particular, the longevity of Insulin/IGF-1 receptor-deficient mutants (daf-2(−)) has been well known as an aging regulation pathway evolutionarily well conserved in other higher subjects such as Drosophila, mice, and the like including Caenorhabditis elegans. Such Insulin/IGF-1 signaling pathways are well conserved in Caenorhabditis elegans and the discovery of constitutional factors of insulin signaling pathways at important protein and DNA levels has actually been achieved using Caenorhabditis elegans.

However, mechanisms for regulation of lifespan by sensory neurons have not yet been discovered and, in particular, technologies for lifespan regulation through sensory neurons have never been developed.

### SUMMARY OF THE INVENTION

Therefore, on the premise that gene manipulation of Caenorhabditis elegans is easy and Caenorhabditis elegans may be a good animal model to discover aging-related mechanisms and pathological roles, the inventors of the present disclosure have completed a Caenorhabditis elegans animal model, the lifespan of which may be regulated by over-expressing a channelrhodopsin protein in ASI sensory neurons and controlling the activation of the ASI sensory neurons by light having a particular wavelength.

One or more embodiments include transgenic Caenorhabditis elegans including, in sensory neurons, Channelrhodopsin 2 (ChR2):: Green Fluorescence Protein (GFP) DNA in which the ChR2 gene and the GFP gene are linked, a method of producing the same, a method of regulating the lifespan thereof, and a method of screening an aging regulation candidate by using the same.

Additional aspects will be set forth in part in the description which follows and, in part, will be apparent from the description, or may be learned by practice of the presented embodiments.

According to an aspect of the present disclosure, there is provided transgenic Caenorhabditis elegans including, in sensory neurons, Channelrhodopsin 2 (ChR2)::Green Fluorescence Protein (GFP) DNA in which the ChR2 gene and the GFP gene are linked.

In one embodiment, the transgenic Caenorhabditis elegans is an animal model for research into aging-related diseases.

In another embodiment, the aging-related diseases include diabetes, obesity, arteriosclerosis, and hypertension.

In still another embodiment, the sensory neurons are activated by blue light stimulation and all-trans retinal (ATR) treatment.

In yet another embodiment, the activation of the sensory neurons is induced by opening the channel of the ChR2 protein.

In yet another embodiment, the lifespan of the transgenic Caenorhabditis elegans is decreased by blue light stimulation and ATR.

In yet another embodiment, the lifespan of the transgenic Caenorhabditis elegans is decreased by inactivation of DAF-16 (FOXO transcription factor).

According to another aspect of the present disclosure, a method of producing the transgenic Caenorhabditis elegans includes: (a) constructing a recombinant vector comprising ChR2::GFP DNA, and (b) injecting the recombinant vector into Caenorhabditis elegans.

In one embodiment, in process (a), the recombinant vector is constructed such that ChR2 gene and GFP gene are introduced into a vector having a str-3 promoter and cloned.

In another embodiment, in process (b), the recombinant vector is injected into Caenorhabditis elegans by microinjection.

According to still another aspect of the present disclosure, a method of regulating the lifespan of the transgenic Caenorhabditis elegans includes controlling activation of the sensory neurons.

In one embodiment, the activation of the sensory neurons is induced by blue light stimulation and ATR treatment.

In another embodiment, the lifespan is decreased by inducing the activation of the sensory neurons.

In still another embodiment, the lifespan is decreased by inactivation of DAF-16 (FOXO transcription factor). According to yet another aspect of the present disclosure, there is provided a method of screening an aging regulation candidate by using the transgenic Caenorhabditis elegans.

In one embodiment, the method includes: (a) subjecting the transgenic Caenorhabditis elegans described above to blue light stimulation and ATR treatment; (b) treating the resulting transgenic Caenorhabditis elegans with an aging regulation candidate; (c) measuring lifespan changes after the treating; and (d) selecting candidates exhibiting an increase in the lifespan changes as compared to a control not treated with the aging regulation candidate.

In another embodiment, the method includes: (a) subjecting the transgenic Caenorhabditis elegans described above to blue light stimulation and ATR treatment; (b) treating the resulting transgenic Caenorhabditis elegans with an aging regulation candidate; and (c) selecting candidates having an increased activity of DAF-16 (FOXO transcription factor) as compared to a control not treated with the aging regulation candidate.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a view illustrating an expression vector for over-expression of a channelrhodopsin in ASI sensory neurons of Caenorhabditis elegans and a process of producing a transgenic animal;

FIG. 2 is a green fluorescent image of transgenic Caenorhabditis elegans in which a GFP-linked channelrhodopsin is expressed in ASI sensory neurons;

FIG. 3 is a view illustrating a process of excitation of cell membrane potential by introduction of cations through opening of the channel of a channelrhodopsin by blue light in the presence of all-trans retinal (ATR), which is a co-factor;

FIG. 4 illustrates green fluorescent images of Caenorhabditis elegans expressing GFP-linked DAF-16 (FOXO transcription factor) and an ASI sensory neuron-specific channelrhodopsin, from which is confirmed that, in about 30 minutes after activation of ASI sensory neurons with blue light and ATR, most of the DAF-16::GFP in the nucleus is transferred to the cytoplasm and thus is inactivated therein;

FIG. 5 is a graph showing measurement results according to time period of a proportion of Caenorhabditis elegans with DAF-16::GFP observed in nuclei thereof after exposure to blue light together with ATR treatment, to confirm changes in the activity of DAF-16 according to time; and

FIG. 6 is a graph showing the lifespan of Caenorhabditis elegans expressing an ASI sensory neuron-specific channelrhodopsin, from which is confirmed that the lifespan of Caenorhabditis elegans was significantly decreased when ASI sensory neurons were activated with light under food-deprived conditions.

### DETAILED DESCRIPTION OF EXEMPLARY EMBODIMENTS

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the present disclosure.

Transgenic Caenorhabditis elegans according to an embodiment of the present disclosure includes, in sensory neurons, Channelrhodopsin 2 (ChR2)::Green Fluorescence Protein (GFP) DNA in which the ChR2 gene and the GFP gene are linked.

The transgenic Caenorhabditis elegans has been designed as an animal model in which the activity of insulin signaling pathways and subjects' lifespans may be regulated by over-expressing a channelrhodopsin protein in ASI sensory neurons and controlling the activation of the ASI sensory neurons with light having a particular wavelength.

The term “Caenorhabditis elegans” used as a scientific name herein refers to nematodes which consume bacteria in soil and are widely used in genetics, molecular biology, and cell biology experiments.

The term “transgenic” as used herein refers to partial transformation of animal's traits by artificial insertion of a recombinant exogenously introduced gene into the animal's chromosome.

The term “transgenic Caenorhabditis eleganlys” as used herein refers to Caenorhabditis elegans including, in sensory neurons, ChR2::GFP DNA constructed by linking the ChR2 gene to the GFP gene.

In the present disclosure, a channelrhodopsin is a light-gated cation channel and seven transmembrane α-helix protein, and Green Fluorescence Protein (GFP) is a protein expressed in cells and emit strong green fluorescence and thus, due to easy observation thereof, is used in various fields that measure whether or not genes are expressed.

Meanwhile, a central nervous system of nematodes is a nerve ring surrounding a middle part of the esophagus, and a nerve trunk extends from anterior and posterior parts of the nerve ring and is divided into several branches at anterior and posterior ends of the worm. A sensory receptor for chemical components, positioned at a front end of the worm is referred to as an amphid, and sensory neurons, such as ASI, ADF, ASG, ASJ, and the like, are present in the amphid.

The transgenic Caenorhabditis elegans may be used as an animal model for aging-related diseases. In this regards, the aging-related diseases may include, for example, metabolic diseases such as diabetes, obesity, arteriosclerosis, hypertension, and the like, or aging, but the present disclosure is not limited thereto.

In the transgenic Caenorhabditis elegans, the membrane channel of a channelrhodopsin protein is opened by blue light stimulation and all-trans retinal (ATR) treatment and thus cell membrane potential thereof is excited, resulting in activation of sensory neurons. In this regard, the blue light refers to light having a wavelength of ˜470 nm, and ATR is a cofactor needed for opening the channel of a channelrhodopsin.

By the blue light stimulation and the ATR treatment, DAF-16 of the transgenic Caenorhabditis elegans is inactivated and thus the lifespan thereof is decreased. DAF-16, which is an FOXO-family transcription factor, acts as a downstream signal of an insulin signaling pathway in Caenorhabditis elegans.

A method of producing the transgenic Caenorhabditis elegans, according to another embodiment of the present disclosure includes: (a) constructing a recombinant vector including ChR2::GFP DNA; and (b) injecting the recombinant vector into Caenorhabditis elegans.

The term “recombinant vector” as used herein refers to a vector used in genetic engineering. The recombinant vector may be a plasmid vector, but the present disclosure is not limited thereto. For example, a viral vector, a cosmid vector, a bacterial artificial chromosome (BAC), a yeast artificial chromosome (YAC), and other non-plasmid vectors may also be used.

The recombinant vector may further include a marker gene for identifying transformation. In this regard, the marker gene is not particularly limited, but may be a fluorescent protein gene such as a green fluorescent protein (GFP), a red fluorescent protein (RFP), or the like.

In addition, the recombinant vector may further include a promoter for protein expression. The term “promoter” as used herein refers to a DNA sequence which regulates the expression of a nucleic acid sequence operably linked to a particular host cell, and the promoter may be any promotor used for expression without particular limitation, but preferably is a str-3 promoter known to be expressed only in ASI sensory neurons of Caenorhabditis elegans. In addition, the term “operably linked” as used herein refers to linking of one nucleic acid fragment to another nucleic acid fragment and thus function or expression thereof is affected by the other nucleic acid fragment.

In addition, the recombinant vector may further include any operator sequence for regulation of transcription, a sequence encoding an appropriate mRNA ribosome binding site, and a sequence regulating termination of transcription and translation.

Genes encoding ChR2 and GFP, introduced into the recombinant vector may be arranged to coincide with a transcription direction of promoters present on the vector so as to effectively induce expression of the genes by activity of the respective promoters. In this regard, the ChR2 gene may be positioned at an N-terminal or C-terminal of the GFP gene, preferably, at a C-terminal of the GFP gene to effectively express a fusion protein.

The injecting of the recombinant vector into Caenorhabditis elegans may be performed using any method known in the art without limitation, preferably, by microinjection using a micropipette.

A method of regulating the lifespan of the transgenic Caenorhabditis elegans, according to another embodiment of the present disclosure includes controlling the activation of sensory neurons. According to the method, the transgenic Caenorhabditis elegans including ChR2::GFP DNA in sensory neurons is subjected to blue light stimulation and ATR treatment to activate the sensory neurons thereof, thereby decreasing the lifespan thereof.

According to another embodiment of the present disclosure, there is provided a method of screening an aging regulation candidate by using the transgenic Caenorhabditis elegans including ChR2::GFP DNA in sensory neurons.

The screening method may include: (a) subjecting the transgenic Caenorhabditis elegans to blue light stimulation and ATR treatment; (b) treating the resulting transgenic Caenorhabditis elegans with an aging regulation candidate; (c) measuring lifespan changes after the treatment; and (d) selecting candidates exhibiting an increase in the lifespan changes as compared to a control not treated with the aging regulation candidate.

The screening method may include: (a) subjecting the transgenic Caenorhabditis elegans to blue light stimulation and ATR treatment; (b) treating the resulting transgenic Caenorhabditis elegans with an aging regulation candidate; and (c) selecting candidates having an increased activity of DAF-16 as compared to a control not treated with the aging regulation candidate.

According to the screening method, aging regulation genes or drugs may be rapidly and easily searched for, which may significantly contribute to the development of new drugs for targeting various aging-related metabolic diseases (e.g., diabetes, obesity, arteriosclerosis, hypertension, and the like).

Hereinafter, exemplary embodiments of the present disclosure will be described in further detail with reference to the following examples. However, these examples are provided only for illustrative purposes and are not intended to limit the scope of the present disclosure.

### EXAMPLE 1

### Culturing of Caenorhabditis elegans

A standard wild-type Bristol strain N2 (Caenorhabditis elegans) was purchased, and then cultured at 20° C. on an E. coli (0P50)-seeded Nematode Growth Medium (NGM) agar plate.

### EXAMPLE 2

### Construction of ChR2::GFP Recombinant Vector

Vector pPD95.75 including the GFP gene was used as a cloning vector for constructing a ChR2:: GFP recombinant vector. First, a str-3 promoter known to be expressed only in ASI sensory neurons of Caenorhabditis elegans and the ChR2 gene were amplified by PCR and then digested with XbaI and KpnI restriction enzymes. The product was inserted into the vector pPD95.75 digested with XbaI and KpnI restriction enzymes.

In this regard, a primer pair for PCR amplification of the ChR2 gene is as follows:

Forward:

5′-GCAGGTCGACTCTAGAGCCACCATGGACTATGGCGG-3′

Reverse:

5′-TCATTTTTTCTACCGGTACCGCTGGCACGGCTCCGGCCTCGGC-3′.

### EXAMPLE 3

### Production of Transgenic Caenorhabditis elegans

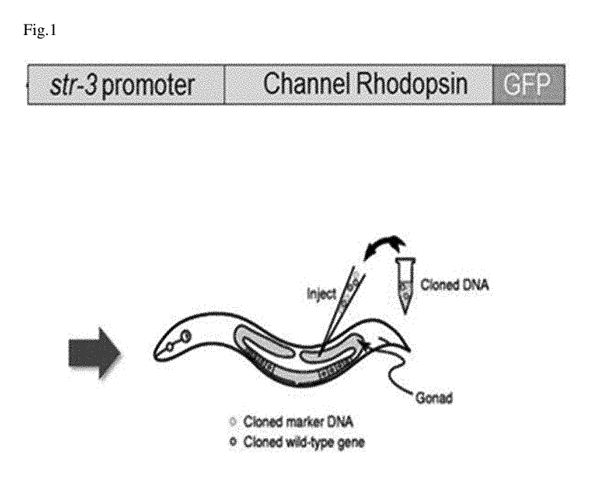

To produce transgenic Caenorhabditis elegans expressing an ASI sensory neuron-specific channelrhodopsin, the ChR2::GFP recombinant vector constructed according to Example 2 was microinjected, using a microinjector, into the genital glands of the wild-type Bristol strain N2 (See FIG. 1).

Subsequently, a single droplet of 2% agarose was dropped on a slide glass, followed by being covered by another slide glass to spread the agarose thereon, and the slide glass used for covering was detached after the agarose was hardened to form an agarose pad. Thereafter, a single droplet of 50 mM NaN₃ was dropped onto the agarose pad and about 15 subjects of the wild-type Bristol strain N2 were transferred thereonto and anesthetized. The agarose pad was cautiously covered by a cover glass so as not to form air bubbles therebetween and then photographed using a fluorescent microscope to observe fluorescence of ChR2::GFP. The Zeiss Axio Scope A1 was used as the fluorescent microscope. The fluorescence shown in the entire body of Caenorhabditis elegans was quantitated using an ImageJ program, and the fluorescence of 20 or more subjects of the wild-type Bristol strain N2 was analyzed through three independent experiments.

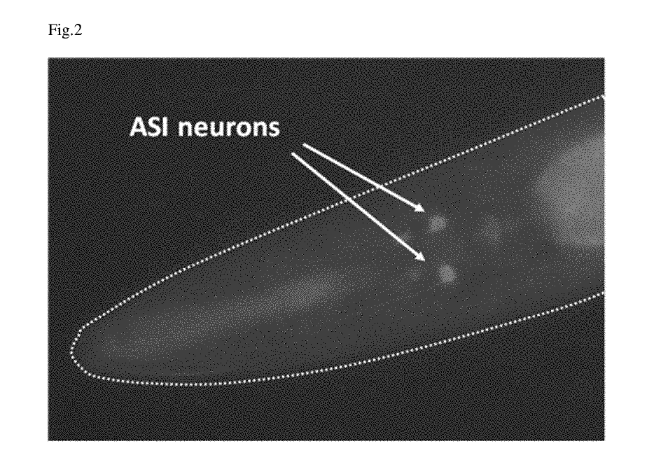

As a result, as illustrated in FIG. 2, it is confirmed that green fluorescence was brightly shown in ASI sensory neurons of the wild-type Bristol strain N2.

### EXAMPLE 4

### Effect of Activation of ASI Sensory Neurons on Activity of DAF-16 (FOXO Transcription Factor)

To confirm an effect of activation of ASI sensory neurons on longevity under food-deprived conditions, the activity of DAF-16, which is a nuclear transcription factor, was evaluated by nuclear/cytoplasmic translocation thereof.

First, the Caenorhabditis elegans (ASI::ChR2) expressing the ASI sensory neuron-specific channelrhodopsin, produced in Example 3 and Caenorhabditis elegans (DAF-16::GFP) expressing GFP-linked DAF-16 were crossbred to obtain Caenorhabditis elegans expressing both ChR2 and DAF-16.

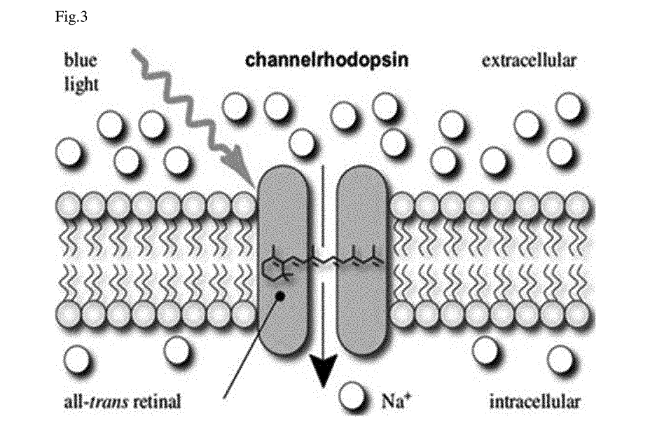

It has been known that the channel of a channelrhodopsin is opened when stimulated with blue light having a wavelength range of ˜470 nm to allow cations to be introduced into the cell, thereby activating neurons, and such a pathway requires all-trans retinal (ATR) as a co-factor (See FIG. 3). Thus, by using such a principle, the activation of ASI sensory neurons was induced by blue light stimulation and ATR treatment, and then an intracellular translocation pattern (nuclear/cytoplasmic translocation) of DAF-16::GFP in enterocytes was evaluated by fluorescence analysis, thereby identifying changes in activity of DAF-16.

In particular, Caenorhabditis elegans expressing DAF-16::GFP and str-3p::ChR2 was food deprived over 24 hours. Subsequently, the corresponding nematode was mounted on an agarose pad and then exposed to blue light, and an intracellular translocation pattern of DAF-16::GFP was analyzed by photographing the corresponding Caenorhabditis elegans over time.

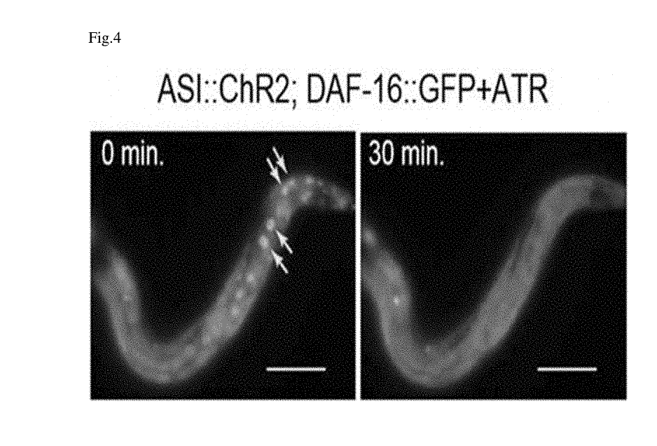

As a result, as illustrated in FIG. 4, it is confirmed that the Caenorhabditis elegans (ASI::ChR2) was food deprived and DAF-16::GFP was translocated (activated) into the nucleus (0 min) and then, when ASI sensory neurons thereof were activated by blue light and ATR treatment, most of the DAF-16::GFP in the nucleus showed cytoplasmic translocation (inactivation) after about 30 minutes (30 min).

In addition, to further identify changes in activity of DAF-16 according to time, Caenorhabditis elegans was exposed to blue light together with ATR, and then a proportion thereof in which DAF-16::GFP was observed in the nucleus was measured according to time period.

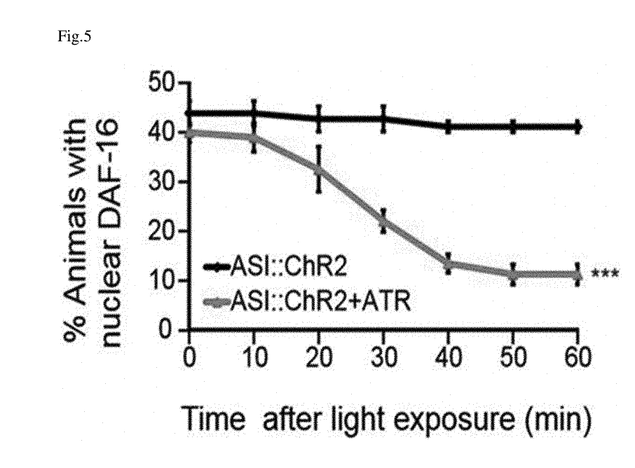

As a result, as illustrated in FIG. 5, it is confirmed that DAF-16 observed in the nucleus gradually decreased as blue light exposure time together with ATR increased (See red solid line). The results indicate that the activity of DAF-16 decreases as the ASI sensory neurons are activated, resulting in shortening of the lifespan thereof.

### EXAMPLE 5

### Control of Subjects' Lifespans by Light

To further experimentally verify whether subjects' lifespans can be regulated by light, based on the results of Example 4, a lifespan measurement experiment was performed on transgenic Caenorhabditis elegans expressing an ASI sensory neuron-specific channelrhodopsin.

That is, to identify an increase in the lifespan of the transgenic Caenorhabditis elegans under food-deprived (FD) conditions rather than under abundantly fed conditions, it was tested whether lifespan changes occurred when the activation of ASI sensory neurons was induced by blue light.

In particular, eggs of adult Caenorhabditis elegans expressing str-3p:: ChR2 were laid for about 12 hours and then the eggs were hatched and grown. On day 1 of adulthood, the Caenorhabditis elegans adults were divided into a total of four experimental groups (fed conditions, fed conditions with exposure to blue light, food-deprived conditions, and food-deprived conditions with exposure to blue light) and were moved on new agar plates. A total of 120 to 150 Caenorhabditis elegans adults was used for each experimental group and exposed to blue light for 2 minutes for each plate at an interval of 24 hours. Once every three days, nematodes moving when given physical stimulation by platinum wires on the plate were counted as alive and nematodes not moving when given the same stimulation were counted as dead.

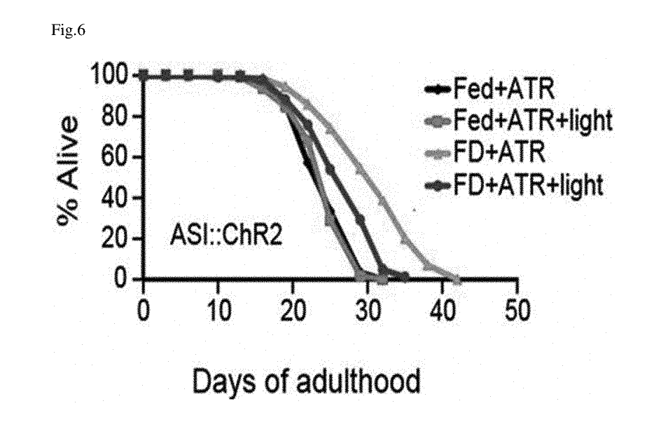

As a result, as illustrated in FIG. 6, it is confirmed that, based on the result of no lifespan difference between ‘Fed+ATR’ and ‘Fed+ATR +light’, inducing the activation of ASI sensory neurons by light barely affects lifespans under abundantly fed conditions.

However, it is confirmed that, based on the result of a considerable lifespan difference between ‘FD+ATR’ and ‘FD+ATR +light’, a significant decrease in lifespan was shown by activation of ASI sensory neurons by light under food-deprived conditions.

The results indicate that a decrease in the activation of sensory neurons contributes to the increase in lifespan under food-deprived conditions and also indicates that artificially inducing the activation of sensory neurons leads to a decrease in lifespan.

Thus, according to the present disclosure, there may be provided a tool capable of regulating lifespan by subjects' insulin signaling pathways by controlling the activation of sensory neurons by light by using a channelrhodopsin protein of the sensory neurons.

The transgenic Caenorhabditis elegans according to the present disclosure has been designed to over-express a channelrhodopsin protein in ASI sensory neurons and thus control the activation of the sensory neurons by light having a particular wavelength. That is, it is confirmed that, by specifically expressing, in ASI sensory neurons of Caenorhabditis elegans, a channelrhodopsin capable of exciting cell membrane potential through opening the channel thereof by light having a particular wavelength, the activation of the sensory neurons is regulated and thus the activity of insulin signaling pathways such as DAF-16 in various internal body tissues may be controlled and subjects' lifespans may also be regulated.

Thus, according to the present disclosure, research into technologies for controlling the activity of insulin signaling pathways and subjects' lifespan by using light may be conducted.

The present disclosure may also provide an animal model for research into prevention/treatment of aging-related diseases by regulating the lifespan of an animal on a subject level.

The present disclosure may also provide a method of screening a drug candidate for prevention/treatment of aging-related diseases by regulating the lifespan of an animal on a subject level.

Although the present disclosure has been described with reference to exemplary embodiments, it will be understood by one of ordinary skill in the art to which the invention pertains that various changes in form and details may be made without departing from the spirit or essential characteristics of the invention. Therefore, the embodiments set forth herein are provided only for illustrative purposes and are not intended to limit the scope of the invention.

What is claimed is:

1. Transgenic Caenorhabditis elegans comprising, in sensory neurons, Channelrhodopsin 2 (ChR2)::Green Fluorescence Protein (GFP) DNA in which the ChR2 gene and the GFP gene are linked.

2. The transgenic Caenorhabditis elegans of claim 1, wherein the transgenic Caenorhabditis elegans is an animal model for research into aging-related diseases.

3. The transgenic Caenorhabditis elegans of claim 2, wherein the aging-related diseases comprises diabetes, obesity, arteriosclerosis, and hypertension.

4. The transgenic Caenorhabditis elegans of claim 1, wherein the sensory neurons are activated by blue light stimulation and all-trans retinal (ATR) treatment.

5. The transgenic Caenorhabditis elegans of claim 4, wherein the activation of the sensory neurons is induced by opening a channel of the ChR2 protein.

6. The transgenic Caenorhabditis elegans of claim 1, wherein a lifespan of the transgenic Caenorhabditis elegans is decreased by blue light stimulation and ATR.

7. The transgenic Caenorhabditis elegans of claim 6, wherein the lifespan of the transgenic Caenorhabditis elegans is decreased by inactivation of DAF-16 (FOX( )transcription factor).

8. A method of producing the transgenic Caenorhabditis elegans of claim 1, the method comprising:
constructing a recombinant vector comprising ChR2:: GFP DNA; and
injecting the recombinant vector into Caenorhabditis elegans.

9. The method of claim 8, wherein, in the constructing, the recombinant vector is constructed such that the ChR2 gene and the GFP gene are introduced into a vector having a str-3 promoter and cloned.

10. The method of claim 8, wherein, in the injecting, the recombinant vector is injected into Caenorhabditis elegans by microinjection.

11. A method of regulating a lifespan of the transgenic Caenorhabditis elegans of claim 1, the method comprising controlling activation of the sensory neurons.

12. The method of claim 11, wherein the activation of the sensory neurons is induced by blue light stimulation and ATR treatment.

13. The method of claim 12, wherein the lifespan is decreased by inducing the activation of the sensory neurons.

14. The method of claim 13, wherein the lifespan is decreased by inactivation of DAF-16 (FOXO transcription factor).

15. A method of screening an aging regulation candidate by using the transgenic Caenorhabditis elegans of claim 1.

16. The method of claim 15, wherein the method comprises:
subjecting the transgenic Caenorhabditis elegans of claim 1 to blue light stimulation and ATR treatment;
treating the resulting transgenic Caenorhabditis elegans with an aging regulation candidate;
measuring lifespan changes after the treating; and
selecting candidates exhibiting an increase in the lifespan changes as compared to a control not treated with the aging regulation candidate.

17. The method of claim 15, wherein the method comprises:
subjecting the transgenic Caenorhabditis elegans of claim 1 to blue light stimulation and ATR treatment;
treating the resulting transgenic Caenorhabditis elegans with an aging regulation candidate; and
selecting candidates having an increased activity of DAF-16 (FOXO transcription factor) as compared to a control not treated with the aging regulation candidate.
